(19) United States
(12) Patent Application Publication
(10) Pub. No.: US 2004/0117862 A1
(43) Pub. Date: Jun. 17, 2004

(54) Production of high levels of transgenic factor VII with engineered stability and its therapeutic uses

(76) Inventors: Julian D. Cooper, Blacksburg, VA (US); William Hugold Velander, Blacksburg, VA (US); Stephen P Butler, Blacksburg, IN (US)

Correspondence Address: Crawford Maunu, Suite 390, 1270 Northland Drive, St Paul, MN, 55120, US

(21) Appl. No.: 10/471,493

(22) PCT Filed: Mar. 11, 2002
(86) PCT No.: PCT/US02/07530

(51) Int. Cl. A01K 67/027

(52) U.S. Cl. 800/7; 800/14

(57) ABSTRACT

A non-human trangenic mammalian animal, as described above, contains an exogenous double stranded DNA sequence stably integratedintot he gemone of the animal, which comprises cis-acting regulatory untis operably linked to a DNA sequence encoding a modifed or variant human FVIII protein, and a signal sequence and secretion motif that is active in directing newly expressed Factor VIII into the milk of the animal at levels an in an unactivated, nondegraded and otherwise stable form that is suitable for subsequent processing for therapeutic applications in treating Hemophilia A. The transgenic mammals are preferably pigs, cows, sheep, goats and rabbits. The applications include milk derivatives used for oral delivery and oral tolerization in the treatment of Hemophilia A.

### FIELD OF THE INVENTION

The invention provides, among other things, a system for producing transgenic proteins, compositions comprising transgenic proteins, transgenic organisms for making proteins, for modifying transgenic proteins in vivo. Illustrative embodiments of the invention particularly provide transgenic animals that express an exogenous gene for vitamin K-dependent proteins, protease inhibitors, blood clotting proteins and mammalian relaxins. In a highly particular illustrative embodiment in this regard the invention provides transgenic female pigs that express these same proteins in their milk in a temporally controlled manner during lactation using a multi-gene inducible system. In this regard, the invention relates particularly to female pigs having stably incorporated in their genomes non-endogenous DNA comprising a region that encodes these same proteins operably linked to a multi-gene system containing at least two different promoters in separate DNA constructs, where one of these promoters is a non-mammary gland specific promoter. Further in this regard the invention relates to the milk containing these same proteins and corresponding compositions derived from the milk. And it also relates to, among other things, uses of these proteins in wellness and therapeutic applications.

### BACKGROUND

The concept of producing important pharmaceutical and nutriceutical proteins in transgenic animals is now firmly established (Van Cott, K. E. and Velander, W. H., Exp. Opin. Invest. Drugs, 7(10): 1683-1690 (1998)), with three potential products, alpha-1 antitrypsin, antithrombin III and alpha glucosidase in the late stages of clinical trials. These proteins, and nearly all other transgenic polypeptides being developed commercially, were produced from a single DNA construct designed to produce a single polypeptide. In general terms, this “classical” design incorporates three distinct regions of DNA, which are all joined or operably linked in one contiguous strand.

The first region of DNA is a tissue specific promoter, in the above mentioned examples a milk protein promoter, which directs expression of the gene to a target organ, the mammary gland, which is regulated by lactogenic hormones, growth factors, cell-cell and cell-substratum interactions. The second region of DNA is the coding region, which may consist of complimentary DNA (cDNA, containing no introns), genomic DNA (gDNA) or a combination of both in a format called a mini-gene. It is important to note that cDNAs, and perhaps also mninigenes, have a silencing effect (failure to express or poor expression levels) on adjacent transgenes (Clark, A. J., et al., NAR, 25(5), 1009-1014, 1997). Therefore, a method of overcoming this silencing effect using non-genomic DNA sequences is highly desirable. The coding region contains the information needed to produce a specific protein, including any processing and secretory signals. The third region, the 3′ region, contains further regulatory sequences and may influence the quantity of polypeptide that is produced from that construct. Non-genomic DNA sequences are inherently smaller than gDNA sequences and are therefore, much easier to manipulate in classical transgene formats.

Although this classical design has been successful in producing commercially viable quantities of certain proteins, there are two areas in which this system is not optimal. First, it is generally accepted that using cDNAs or minigenes in a classically designed construct, is less efficient for protein production than using a corresponding gDNA coding region. Indeed, this is such a problem that methods have been developed to address this issue (Clark, A. J. et al, Biotechnology 10, 1450-1454, 1992). Whilst these methods can improve the efficiency and level of expression of cDNAs and minigenes to some extent, they do not improve expression to the same level as is typically obtained using gDNA. A higher level would be ideal for commercial protein production.

The second area in which the classical single gene DNA construct design is suboptimal is in the production of highly biologically active proteins in transgenic animals. Proteins with an extremely high biological activity can be detrimental to the transgenic animal, even if circulatory levels (or other systemic levels) are low (Castro, F. O., et al., Selection of Genes for Expression in Milk: The Case of the Human EPO Gene, in Mammary Gland Transgenesis. Therapeutic Protein Production. Castro and Janne (eds.) Springer-Verlag Berlin New York, 91-106, 1998). This can be due in large part to either ectopic expression (expression of the transgene in organs other than the targeted one) or leakage of the protein product into the blood from the target organ. If the protein product is highly biologically active, expression ideally must be strictly controlled so that the animal is exposed to the product for a short time only, thus reducing the chance of any lasting detrimental effects. This requires an expression system that can be turned on and off very rapidly and precisely.

Regulation of Promoters

The expression of many genes is controlled at the level of transcription, when the DNA sequences are transcribed into RNA, prior to being translated into protein (Latchman, D. S., Eukaryotic Transcription Factors, Academic Press, 1998). The DNA sequence element that controls transcription is the promoter. This generally contains a small core region, which is capable of directing constitutive or basal levels of transcription, and upstream response elements that control spatial and temporal regulation of transcription. These DNA sequences include two types of elements, those which are involved in the basic process of transcription and are found in many genes exhibiting distinct patterns of regulation, and those found only in genes transcribed in a particular tissue or in response to a specific signal. The latter elements likely produce this specific expression pattern. They are binding sites for a wide range of different cellular proteins (transcription factors) whose levels fluctuate in response to stimuli from external or internal sources. Gene expression in a given tissue may be stimulated or inhibited depending on the type and amount of transcription factors that are present in that tissue at any time. Many transcription factors or other proteins that enable transcription factor pathways are largely uncharacterized from the perspective of an exact biochemical analysis, which details their conformationally-dependent interactions with DNA. Overall, the regulation of expression at the DNA level, is a function of which regulatory elements (binding sites) are present in the promoter and how the cell or tissue responds to its environment by changing the relative levels of the different DNA binding transcription factors in the cell. e

Another mechanism involved in the precise control of gene expression is transcriptional repression (Maldonado, E., et al, Cell, 99(5), 455-458, 1999). Transcriptional repressor proteins associate with their target genes either directly through a DNA-binding domain or indirectly by interacting with other DNA-bound proteins. The repressor protein can inhibit transcription by masking a transcriptional activation domain, blocking the interaction of an activator with other transcription components or by displacing an activator from the DNA.

Milk protein genes are characterized by a strict tissue specific expression and regulation during the process of functional differentiation. They are coordinately expressed in response to various developmental signals, such as changing levels of lactogenic hormones (prolactin, insulin, glucocorticoids, progesterone), local levels of certain growth factors (EGF), cell-cell interactions and interactions with extra-cellular matrix (ECM) components (Rijnkels, M. and Pieper, F. R., Casein Gene-Based Mammary Gland-Specific Transgene Expression, in Mammary Gland Transgenesis. Therapeutic Protein Production. Castro and Janne (eds.) Springer-Verlag, Berlin, New York, 41-64, 1998).

Lactogenic hormones activate latent transcription factors in the cytoplasm of mammary epithelial cells. The steroid hormones progesterone, estrogen, and glucocorticoid regulate the transcription of target genes by binding to specific intracellular receptors. Some models purport that binding of the hormone with its receptor changes the receptor's conformation from a physiologically inactive form to a form which is active and capable of dimerization. The active receptors are then capable of binding specific DNA sites in the regulatory region of the target gene promoters, stimulating gene transcription and thus, protein synthesis. Steroid receptors belong to a superfamily of ligand-inducible transcription factors and it has been well documented that these are modular proteins organized into structurally and functionally defined domains. It has also been shown that these domains can be rearranged as independent cassettes within their own molecules or as hybrid molecules with domains from other regulatory peptides. Interestingly, the transactivation domains of the glucocorticoid receptor can be duplicated in tandem and show positional independence in a “super receptor” with 3-4 times the activity of the wild type protein. (Hollenberg, S. M. and Evans, R. M., Cell, 55, 899-906, 1988; Fuller, P. J., FASEB J., 5, 3092-3099, 1991; U.S. Pat. No. 5,364,791; U.S. Pat. No. 5,935,934; Whitfield, G. K., et al, J.Cell.Biochem., suppl. 32-33, 110-122, 1999; Braselmann, S., et al, PNAS, 90, 1657-1666, 1993). The structure and function of the steroid receptor superfamily is well conserved. Generally there are three main domains and several sub-domains or regions. The NH2-terminal domain is the least conserved in size and sequence and contains one of the two, transactivation sequences of the receptor. The central DNA binding domain of about 70 amino acids is highly conserved, as is the COOH-terminal ligand binding domain. This latter domain also contains sub-domains responsible for dimerization, heat shock protein (hsp) 90 binding, nuclear localization and transactivation.

Prolactin plays the essential role in milk protein gene expression and exerts its effect through binding to the extracellular domain of the prolactin receptor and through receptor dimerization. This activates a protein tyrosine kinase (JAK2) which is non-covalently associated with the cytoplasmic domain of the prolactin receptor (Gouilleux, F., et al, EMBO J., 13(18), 4361-4369, 1994; Imada, K. and Leonard, W. J., Mol. Immunol., 37(1-2), 1-11, 2000). The activated JAK2 phosphorylates the signal transducer and transcription activator, Stat 5, causing it to dimerize and subsequently, translocate to the nucleus. Once in the nucleus, Stat5 specifically binds to sequence elements in the promoter regions of milk protein genes (Liu, X., et al, PNAS, 92, 8831-8835, 1995; Cella, N., et al, Mol.Cell.Biol., 18(4), 1783-1792, 1998; Mayr, S., et al, Eur.J.Biochem., 258(2), 784-793, 1998). In an analysis of 28 milk protein gene promoters (Malewski, T., BioSystems, 45, 29-44, 1998) there were 4 transcription factor binding sites that were present in every promoter, C/EBP, CTF/NF1, MAF and MGF (Stat 5). Although steroid hormone receptors and Stat factors comprise two distinct families of inducible transcription factors their basic structure is similar. Stat proteins are modular with an amino terminus that regulates nuclear translocation and mediates the interaction between Stat dimers (Callus, B. A. and Mathey-Prevot, B., J.Biol.Chem., 275(22), 16954-16962, 2000). There is a central DNA binding domain and a carboxy terminal region, which contains the phosphorylation site and a transactivation domain.

Egg white genes seem to be regulated in a similarly complex manner. It is known that the progesterone-dependent activation of the egg white genes in the chicken oviduct is mediated through the progesterone receptor (Dobson, A. D. W., et al, J.Biol.Chem., 264(7), 4207-4211, 1989). In addition, the chicken ovalbumin upstream promoter-transcription factor (COUP-TF) is a high affinity and specific DNA binding protein, which interacts as a dimer with the distal promoter sequence of the ovalbumin gene and promotes initiation of transcription of this gene by RNA polymerase (O'Malley, B. W. and Tsai, M -J., Biol.Reprod., 46, 163-167, 1992). COUP-TFs are orphan members (no binding ligand has as yet been determined for these receptors) of the nuclear receptor superfamily, and have been shown to play a key role in the regulation of organogenesis, neurogenesis, metabolic enzyme production and cellular differentiation during embryogenic development, via transcriptional repression and activation (Sugiyama, T., et al, J.Biol.Chem., 275(5), 3446-3454, 2000).

A protein expression method based on the inducible Tet repressor system has been developed (Furth, P. A., et al, PNAS, 91, 9302-9306, 1994), but the levels of basal expression without induction are too high to be useful in transgenic animals (Soulier S. et al, Eur. J. Biochem. 260, 533-539, 1999). Another inducible system based on the use of the ecdysone receptor has been reported (No, D., et al, PNAS, 93, 3346-3351, 1996; PCT 97/38117, PCT 99/58155) and has recently given encouraging results in transgenic mice (Albanese, C., et al, FASEB J., 14, 877-884, 2000). However, this system required the delivery of an exogenous ligand to the mice for the full lactation period. Such a ligand would be costly and difficult to procure for regular administration in a production environment.

A new multi-gene system for protein production in transgenic animals would improve commercial levels of production from cDNA constructs by amplifying specifically tailored transcription factors which need not naturally occur in the tissue targeted for expression, but would be transgenically expressed specifically in that tissue. Unlike classical gene expression formats for recombinant proteins, the tissue specific promoter would not be linked to the protein to be expressed, but would be used to drive expression of transcription factors which do not have a signal sequence and so are not secreted. In addition, the added control that a doubly inducible multi-gene system would provide, which is inexpensive and easily applied, could enable the production of highly biologically active proteins in transgenic animals in a pulsatile fashion so as to avoid longterm detrimental effects.

Proteins for Transgenic Production

A multi-gene system, as described below, can be used to direct expression of any protein, particularly any secreted protein, which can be expressed in a transgenic organism in useful quantities, either for research or commercial development. Particular proteins of interest with respect to production by multi-gene expression systems include relaxin and other hormones with cross-species activity such as growth factors, erythropoitin (EPO) and other blood cell growth stimulating factors. For these proteins, the expression may be problematic in terms of harming the host animal as is known to happen when EPO is expressed for an extended period of time. It is noted that tissue specific expression of transgenes is not an absolute phenomenon and promiscuous expression or systemic transport of the expressed recombinant protein within the animal almost always occurs with any expression system in any animal, albeit at very low levels. However, even at low levels of expression of EPO, when the EPO is expressed over an extended period of time, the hematocrit of the host animal can rise to a fatal level. Thus a temporal control which can enable pulse expression using an external inducer molecule could overcome the problems of continuous and extended expression (ie., as could occur if expression occurs over an entire lactation period). Pulse or truncated expression would be useful in preventing an adverse, systemic physiologic effect by recombinant molecules like EPO, which can cause these effects at very low levels.

Relaxin is widely known as a hormone of pregnancy and parturition and typically circulates at less than 50 pg/ml in the blood of women. However, it is now emerging that the peptide has a far wider biological function than was at first thought. There are receptor sites for relaxin in striated muscle, smooth muscle, cardiac muscle, connective tissue, the autonomic and the central nervous systems. Human relaxin has been demonstrated to inhibit excessive connective tissue build-up and is in Phase II trials for the treatment of Scleroderma Porcine relaxin was available commercially in the 1950-60s and was used extensively for such conditions as cervical ripening, scleroderma, premature labour, PMS, decubital ulcers and glaucoma. Relaxin is known to adversely affect the lactation of different mammalian species but does not seem to affect the pig in a similar manner. Therefore, the pig is perfectly suited for production of relaxin in milk.

Other examples of proteins which it would be desirable to produce in transgenic organisms, are proteins that are protease inhibitors. Some examples of protease inhibitors are Alpha 1-antitrypsin, Alpha 2 Macroglobulin, and serum leukocyte protease inhibitor. These proteins are serine protease inhibitors that show antiviral, non-steroidal anti-inflammatory and wound healing properties. These proteins are useful in veterinary, cosmetic and nutriceutical applications.

Alpha 1-antitrypsin (AAT) is a naturally occurring glycoprotein produced by the liver. Improperly glycosylated recombinant AAT such as made by yeast, does not have a sufficient circulation half-life to be used as a parenterally administered therapeutic. Congenital deficiency results in the condition emphysema and in 1985 Bayer Pharmaceuticals began marketing a plasma derived AAT product, Prolastin. Unfortunately, due to shortages of Asafe@ plasma and frequent recalls, supplies of Prolastin are often very limited. AAT has also been used to treat psoriasis, atopic dermatitis, ear inflammation, cystic fibrosis and emphysema, and to assist in wound healing. It has been estimated that over 10 million people in the US alone may benefit from AAT therapies.

Alpha 2 macroglobulin (A2M) is a very large, complex glycoprotein with a published cDNA sequence containing 1451 amino acids. The mature protein is a tetrameric molecule composed of four 180 kDa subunits and thus has a molecular weight which is over 720 kDa. Its complexity makes it most suited for production in mammalian systems but few mammalian systems will likely make A2M at commercially viable levels. A2M is indicated for treatment of asthma, bronchial inflammation and eczema and acts as a protease inhibitor to both endogenous and exogenous proteases that cause inflammation. A2M is necessarily more potent than alpha 1-antitrypsin due to its irreversible binding of target proteases. A2M is also useful in inhibiting proteases frequently found in (thermal) burn wounds from yeast and other infections. The high specific activity of these types of proteases allows for smaller doses during treatment. Thus, A2M=s complexity and specific activity make it ideally suited for production in transgenic pig mammary glands.

Vitamin K-Dependent Proteins

Vitamin K-dependent (VKD) proteins such as those proteins associated with haemostasis have complex functions which are largely directed by their primary amino acid structure. In particular, the post-translational modification of glutamic acids in the amino terminal portion of these molecules is essential for proper biological activity. This includes biological activity of both pro-coagulation and anti-coagulation. This particular domain found in VKD-proteins is called the “gla domain”. For example, the Gla domain is an essential recognition sequence in tissue factor (TF) mediated pro-coagulation pathways. The anti-coagulation of this pathway depends upon the lipoprotein-associated coagulation inhibitor, termed LACI, which is a non-VKD protein. LACI forms a complex with the Gla domain of factor Xa, factor VIIa, and TF. Specifically, the Gla domain of factor Xa (FXa) is needed for this procoagulation inhibitory activity. It has been shown that recombinant chimeric molecules having LACI inhibitor (Kunitz type) regions and the Gla domain of FXa can be inhibitory of the TF pathway.

TABLE 1
VKD proteins.

| | | |
|---|---|---|
| Protein C | Factor X(FX) | Bone Gla protein (Osteocalcin) |
| Protein S | Prothrombin | |
| Protein Z | Factor VII | |
| Factor IX | | |

Gamma-carboxylation is required for calcium-dependent membrane binding. All of the proteins listed in Table 1 have multiple Gla-residues in a concentrated domain. The Gla-domains of these proteins mediate interaction and the formation of multi-protein coagulation protein complexes. Mammalian coagulation (here collectively meaning both pro-coagulation and anti-coagulation pathways and mechanisms) physiology requires that nearly complete-carboxylation of VKD-proteins occurs within the respective Gla domain for each of these proteins to be maximally functional. Notably, in the context of recombinant synthesis of any protein containing Gla-domains, the extent of gamma-carboxylation of VKD-proteins varies from one mammalian cell source to another, including differences between species and tissue within a species.

VKD-proteins of interest with respect to production by single or multi gene expression systems include those in Table 1, particularly blood clotting factor IX, Protein C and chimeric hybrid vitamin K-dependent proteins. Factor IX is an essential blood clotting protein. Haemophilia B is a genetic disorder in which the production of active Factor IX is defective. It is an inherited disorder that primarily affects males, at the rate of approximately 1 in 30,000. The consequent inability to produce sufficient active Factor IX can lead to profuse bleeding, both internally and externally, either spontaneously or from relatively minor injuries.

In spite of techniques available to amplify recombinant synthesis of VKD proteins such as Protein C and Factor IX, biologically functional recombinant versions of these proteins are difficult to produce and are made typically at levels less than about 0.1 grams per liter per 24 hours in recombinant cell culture media (Grinnell, B. W., et al, in Protein C and Related Anticoagulants. Bruley, D. F. and Drohan, W. N. (eds.), Houston, Tex.; Gulf Publishing Company, 29-63, 1990), or less than 0.22 gm per liter per hour in the milk of transgenic livestock (Van Cott, K. E., et al., Genetic Analysis: Biomolecular Eng., 15, 155-160, 1999). The expression of high levels of FIX using a cDNA construct is difficult. However, the gDNA of FIX, at 33 kbp, is rather large and difficult to manipulate, particularly when compared to the FIX cDNA, which is only 1.4 kbp.

Most VKD-blood plasma proteins are also glycosylated. The extent and types of glycosylation observed is heterogeneous and varies considerably in all species and cell types within a species. Examples of the heterogeneity, structure function relationships of glycosylation are cited by Degen, Seminars in Thrombosis and Hemostasis, 18(2), 230-242, 1992; Prothrombin and Other Vitamin K Proteins, Vols I and II, Seegers and Walz, Eds., CRC Press, Boca Raton, Fla., 1986.

Glycosylation is a complex post translational modification that occurs on, many therapeutic proteins. The process of glycosylation attaches polymeric sugar compounds to the backbone of a protein. These sugar-based structures impart not only an immunologically specific signature upon the protein, but also can change the specific level of activity that the protein has with relation to how long it can reside in the bloodstream of a patient, or how active the protein is in its basic function. All three of these facets can make or break the protein in its role as a therapeutic or wellness product. For example, genetically engineered yeast can impart glycosylation that results in an immunologically adverse signature, which can stimulate the body to make antibodies and essentially reject the protein. In fact, that is part of the reason why yeast vaccines are effective; they easily induce an immune response. The mammary gland of ruminants produces a substantial fraction of glycosylation on milk proteins which resemble the primitive sugars found in yeast. Thus, applications that result in the long term, repeated exposure of proteins containing yeast or yeast-like signatures, to human tissue are intensely scrutinized with respect to the potential of adverse immune reactions. This structure is also apt to cause dysfunction with respect to the protein=s natural activity and may also contribute to a shortened residence time in blood. In contrast, the mammary gland of pigs gives a glycosylation signature which more closely resembles that found in normal human blood proteins, helping to assure biochemical function and a long circulatory half-life.

The complex post-translational modifications of therapeutic proteins, such as those discussed above that are necessary for physiological activities, pose a difficult obstacle to the production of active vitamin K dependent proteins in cells using cloned genes. Moreover, attempts to culture genetically altered cells to produce VKD polypeptides have produced uneconomically low yields and, generally, preparations of low specific activity. Apparently, the post-translational modification systems in the host cells could not keep pace with production of exogenously encoded protein, reducing specific activity. Therefore, cell culture production methods have not provided the hoped for advantages for producing highly complex proteins reliably and economically.

An attractive alternative is to produce these complex proteins in transgenic organisms. However, it is likely that only mammals and perhaps birds will be able to carry out all the post-translational modifications necessary for their physiological function. It has not been possible, as yet, to produce commercially viable levels of certain complex polypeptides from a controlled source in a highly active form with a good yield, and there exists a need for better methods to produce such proteins.

An interesting new class of proteins, which is likely to be difficult to produce in commercial quantities in cell culture are the genetically engineered fusion, chimeric and hybrid molecules which are now being developed. These proteins are designed and produced by combining various domains or regions from different natural proteins, either wild type or mutated, which can confer the properties of each domain or region to the final hybrid molecule. An example of this is X_{LC}LACI_{K1} (Girard, T. J., et al., Science 248, 1421-1424, 1990) which is a hybrid protein made up of domains from factor X and lipoprotein-associated coagulation inhibitor (LACI). LACI appears to inhibit tissue factor (TF)-induced blood coagulation by forming a quaternary inhibitory complex containing FXa, LACI, FVIIa and TF. X_{LC}LACI_{K1} directly inhibits the activity of the factor VIIa-TF (tissue factor) catalytic complex, but is not dependent on FXa. Gamma-carboxylation of the FX portion of the hybrid protein is required for inhibitory activity. In order for efficient carboxylation to occur at high levels, it is likely that the pro-peptide of the recombinant VKD-protein must be properly matched to the endogenous carboxylase system (Stanley, T. B., et al, J.Biol.Chem., 274(24), 16940-16944, 1999). This is probably true for all VKD-polypeptides including chimeric ones such as X_{LC}LACI_{K1}. It appears that the endogenous carboxylase systems of any given species or tissue within that species, most of which are not identified or characterized, will differ in their compatibility to any given pro-peptide sequence. Also it is frequently desirable to have the pro-peptide cleaved from the nascent VKD protein, such as a X_{LC}LACI_{K1} polypeptide, once gamma-carboxylation has been completed on the polypeptide's gla domain. It is therefore, also important to find a propeptide sequence that will be efficiently cleaved within the specific species and tissue in which it is being recombinantly produced. These factors render it problematic to find an expression system which can produce desirable amounts of biologically active VKD-proteins such as X_{LC}LACI_{K1} chimeric proteins. In spite of being known as a potent coagulation inhibitor since the early 1990s, X_{LC}LACI_{K1} chimeric molecules have not been made in large amounts in a commercially viable manner (ie., greater than 0.1 gm per liter per 24 hours) in recombinant mammalian cell culture. One way to improve expression of this protein in a transgenic system, particularly in transgenic pigs, may be to substitute the FIX propeptide sequence for the FX propeptide sequence, such a protein would be termed 9XKI.

New therapeutic molecules are being designed to have increased activity, decreased inactivation, increased half-life or specific activity and reduced immunogenicity and/or imrunoreactivity to existing circulating antibodies in patients' bloodstreams. This has been demonstrated in genetically engineered Factor VIII proteins (U.S. Pat. No. 5364771, U.S. Pat. No. 5583209, U.S. Pat. No. 5888974, U.S. Pat. No. 5004803, U.S. Pat. No. 5422260, U.S. Pat. No. 5451521, U.S. Pat. No. 5563045). Mutations include deletion of the B domain (Lind, P., et al., Eur.J.Biochem. 232, 19-27, 1995), domain substitution or deletion, covalent linkage of domains, site-specific replacement of amino acids and mutation of certain cleavage sites. In particular, a genetically engineered inactivation-resistant factor VIII (IR8) has been developed to help in the treatment of hemophilia A (Pipe, S. W. and Kaufman, R. J., PNAS 94, 11851-11856, 1997). The introduction of specific sequences from porcine factor VIII can also be useful in the formation of a recombinant FVIII which is used to treat hemophiliacs with improved properties as stated above. These molecules can also be designed for improved expression. It is widely known that FVIII has restrictions in intracellular trafficking which lead to low levels of secretion. Modification of the domains associated with intracellular interactions with immunoglobulin binding protein (Bip) or calnexin would be examples of modifications used to improve secretory processing efficiency (Kaufman, R. J., Abstract S1-8, 10^{th} Int.Biotech.Symp., Sydney, Australia, 25-30^{th} August., 1996). Factor VIII gDNA is another example of an extremely large and unwieldy DNA sequence (˜110 kbp), whereas the cDNA is only 7 kbp, making it much more manageable.

Whey acidic protein (referred to as “WAP”) is a major whey protein in the milk of mice, rats, rabbits and camels. The regulatory elements of the mouse WAP gene are entered in GenBank (U38816) and cloned WAP gene DNAs are available from the ATCC. The WAP promoter has been used successfully to direct the expression of many different heterologous proteins in transgenic animals for a number a years (EP0264166, Bayna, E. M. and Rosen, J. M., NAR, 18(10), 2977-2985, 1990). Lubon et al (U.S. Pat. No. 5,831,141) have used a long mouse WAP promoter (up to 4.2 kbp) to produce Protein C in transgenic animals. However, the longest rat WAP promoter that has been used is 949 bp (Dale, T. C., et al., Mol.Cell.Biol., 12(3), 905-914, 1992).

### SUMMARY

The present invention is directed to producing transgenic proteins, compositions comprising transgenic proteins, transgenic organisms for making proteins, for modifying transgenic proteins in vivo, and to addressing the previously-discussed issues, e.g., as characterized in connection with the above-cited references each of which is incorporated by reference generally and more specifically as such teachings relate to methodology for related transgenic protein production and applications of such proteins.

In various embodiments of the present invention there is a composition for treating hemophilia A comprising a milk derivative containing recombinant variant of human factor VIII derived from a bodily fluid produced in a transgenic organism as described below.

And in still yet other embodiments of the present invention there is the production of a functional FVIII molecule with a covalently linked A2 subunit and light chain, in a transgenic organism.

And in still yet other embodiments of the present invention there is the production of a functional FVIII molecule with all or most of the B domain deleted, in a transgenic organism.

And in still yet other embodiments of the present invention there is the production of a functional inactivation-resistant form of blood clotting factor VIII with a covalently linked A2 subunit and light chain, in a transgenic organism.

And in still yet other embodiments of the present invention there is a transgenic organism as above where the introduced genetic construct comprises a promoter operatively linked to the region encoding the functional inactivation-resistant form of blood clotting factor VIII, with a covalently linked A2 subunit and light chain, as in the “classical” design for single transgenic constructs, so as to engender production of the functional inactivation-resistant form of blood clotting factor VIII, with a covalently linked A2 subunit and light chain, protein.

And in still yet other embodiments of the present invention there is the production of a functional inactivation-resistant form of blood clotting factor VIII, such as IR8, in a transgenic organism.

And in still yet other embodiments of the present invention there is a transgenic organism as above where the introduced genetic construct comprises a promoter operatively linked to the region encoding the functional inactivation-resistant form of blood clotting factor VIII, such as IR8, as in the “classical” design for single transgenic constructs, so as to engender production of the functional inactivation-resistant form of blood clotting factor VIII protein.

And in still yet other embodiments of the present invention there is a transgenic organism as above where the introduced genetic construct comprises the multi-gene systems A and or B DNA constructs containing the DNA sequence coding for a functional inactivation-resistant form of blood clotting factor VIII, such as IR8, so as to engender production of the functional inactivation-resistant form of blood clotting factor VIII protein.

And in still yet other embodiments of the present invention there is a transgenic organism as above where the introduced genetic construct comprises the multi-gene system A and or B DNA constructs containing DNA sequences coding for a functional inactivation-resistant, human pig hybrid form of blood clotting factor VIII so as to engender production of the functional inactivation-resistant, human pig hybrid form of blood clotting factor VIII protein.

And in still yet other embodiments of the present invention there is a transgenic organism as above where the introduced genetic construct comprises the multi-gene system A and or B DNA constructs containing DNA sequences coding for a functional inactivation-resistant form of blood clotting factor VIII with a covalently linked A2 subunit and light chain so as to engender production of the functional inactivation-resistant form of blood clotting factor VIII, with a covalently linked A2 subunit and light chain, protein.

And in still yet other embodiments of the present invention there is the production of a functional human pig hybrid form of blood clotting factor VIII with a covalently linked A2 subunit and light chain, in a transgenic organism.

And in still yet other embodiments of the present invention there is a transgenic organism as above where the introduced genetic construct comprises a promoter operatively linked to the region encoding the functional human pig hybrid form of blood clotting factor VIII, with a covalently linked A2 subunit and light chain, as in the “classical” design for single transgenic constructs, so as to engender production of the functional human pig hybrid form of blood clotting factor VIII, with a covalently linked A2 subunit and light chain, protein.

And in still yet other embodiments of the present invention there is a transgenic organism as above where the introduced genetic construct comprises the multi-gene system A and or B constructs containing DNA sequences coding for a functional human pig hybrid form of blood clotting factor VIII with a covalently linked A2 subunit and light chain so as to engender production of the functional human pig hybrid form of blood clotting factor VIII, with a covalently linked A2 subunit and light chain, protein.

And in still yet other embodiments of the present invention there is the production of a functional inactivation-resistant, human pig hybrid form of blood clotting factor VIII with a covalently linked A2 subunit and light chain, in a transgenic organism.

And in still yet other embodiments of the present invention there is a transgenic organism as above where the introduced genetic construct comprises a promoter operatively linked to the region encoding the functional inactivation-resistant, human pig hybrid form of blood clotting factor VIII, with a covalently linked A2 subunit and light chain, as in the “classical” design for single transgenic constructs, so as to engender production of the functional inactivation-resistant, human pig hybrid form of blood clotting factor VIII, with a covalently linked A2 subunit and light chain, protein.

And in still yet other embodiments of the present invention there is a transgenic organism as above where the introduced genetic construct comprises the multi-gene system A and or B DNA constructs containing DNA sequences coding for a functional inactivation-resistant, human pig hybrid form of blood clotting factor VIII with a covalently linked A2 subunit and light chain so as to engender production of the functional inactivation-resistant, human pig hybrid form of blood clotting factor VIII, with a covalently linked A2 subunit and light chain, protein.

In other more specific embodiments, the present invention is directed to a non-human transgenic mammal containing an exogenous DNA molecule stably integrated in its genome. The exogenous DNA molecule comprises: (a) a mammary gland-specific gene including a promoter; and (b) a recombinant variant Factor VIII-encoding DNA sequence that encodes an endogenous signal sequence, a Factor VIII pro-sequence and a modified Factor VIII sequence encoding a secretion trafficking motif that is efficient in mammary epithelial cells; and (c) 3′ regulatory sequences from a mammary gland-specific gene, which sequences are operatively linked to said Factor VIII-encoding DNA sequence; and (d) said Factor VIII is stably secreted into the milk at least 20 micrograms modified Factor VIII per milliliter of milk and is not inactivated or degraded by the milk-environment and therefore useable for Factor VIII therapeutic applications.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention may be more completely understood in consideration of the detailed description of various embodiments of the invention in connection with the accompanying drawings, in which:

FIG. 1 is production of the plasmid pUCWAP6SalXma, according to an example embodiment of the present invention;

FIG. 2 is production of the plasmid pUCWAP6IR8(−), according to another example embodiment of the present invention;

FIG. 3 is production of the plasmid pUCWAP6IR8, according to another example embodiment of the present invention.

While the invention is amenable to various modifications and alternative forms, specifics thereof have been shown by way of example in the drawings and will be described in detail. It should be understood, however, that the intention is not necessarily to limit the invention to the particular embodiments described. On the contrary, the intention is to cover all modifications, equivalents, and alternatives falling within the spirit and scope of the invention as defined by the appended claims.

### DETAILED DESCRIPTION

As previously mentioned, the present invention is directed to products and approaches for regulating the expression of a protein in a transgenic organism, methods for obtaining polypeptides from transgenic organisms, compositions comprising transgenically produced polypeptides, and therapeutic uses thereof. For example, one embodiment of the present invention is directed to a non-human transgenic mammal containing an exogenous DNA molecule that is stably integrated in its genome. The exogenous DNA molecule includes a mammary gland-specific gene, a Factor VIII-encoding DNA sequence that performs encoding for applicable sequences, and 3′ regulatory sequences from a mammary gland-specific gene. Surprisingly, in connection with the present invention, it has been discovered that, with the 5′ and 3′ regulatory sequences that are operatively linked to the modified Factor VIII-encoding DNA sequence with the stably integrated exogenous DNA, the modified Factor VIII can be made and secreted into the milk, so that the modified Factor VIII is stable in the milk and can be made suitable for Factor VIII therapeutic applications. Three such applications that are suitable are parenteral Factor VIII therapy using a highly purified FVIII, oral tolerization of this and other Factor VIII therapies prior and during Factor VIII parenteral therapy using milk derivatives from the above mentioned transgenic mammals, and oral delivery of milk derivatives containing variant Factor VIII from the above mentioned transgenic mammals.

The present invention also provides amongst other things, methods for regulating the expression of a protein in a transgenic organism, methods for obtaining polypeptides from transgenic organisms, compositions comprising transgenically produced polypeptides, and uses thereof, as described in greater detail below.

Methods for Making Transgenic Organisms

Transgenic organisms may be produced in accordance with the invention as described herein using a wide variety of well-known techniques, such as those described in Perry, M. M. and Sang, H. M., Transgenic Res. 2, 125-133; Ho Hong, Y. et al., Transgenic Res. 7(4), 247-252, 1998; Genetic Engineering Of Animals, Ed. A. Puhler, VCH Publishers, New York (1993) and in more detail in Volume 18 in Methods in Molecular Biology: Transgenesis Techniques, Eds. D. Murphy and D. A. Carter, Humana Press, Totowa, New Jersey (1993); all of which are incorporated herein by reference in their entireties, particularly as to the foregoing in parts pertinent to methods for making transgenic organisms that express polypeptides. See also for instance Lubon et al., Transfusion Medicine Reviews X(2): 131-141 (1996) and Pursel, V. G., et al., 480 in the proceedings of 11^{th} International Congress on Animal Reproduction and Artificial Insemination, Dublin, Ireland, 1988, which are incorporated herein by reference in their entirety, particularly as to the foregoing in parts pertinent to methods for making transgenic organisms.

In particular, transgenic mammals, such as mice and pigs, that express polypeptides in accordance with certain preferred embodiments of the invention, can be produced using methods described in among others Manipulating The Mouse Embryo, Hogan et al., Cold Spring Harbor Press (1986); Krimpenfort et al., Bio/Technology 9:844 et seq. (1991); Palmiter et al., Cell 42:343 et seq. (1985); Genetic Manipulation of the Early Mamnmalian Embryo, Kraemer et al., Cold Spring Harbor Press, Cold Spring Harbor, N.Y. (1985); Hammer et al., Nature 315: 680 et seq. (1985); U.S. Pat. No. 4,873,191 of Wagner et al. for Genetic Transformation of Zygotes, and U.S. Pat. No. 5,175,384 of Krimpenfort et al. for Transgenic Mice Depleted in Mature T-Cells and Methods for Making Transgenic Mice, each of which is incorporated herein by reference in its entirety, particularly as to the foregoing in parts pertinent to producing transgenic mammals by introducing DNA or DNA:RNA constructs for polypeptide expression into cells or embryos

For example, transgenic organisms of the present invention can be produced by introducing into eggs, or developing embryos, one or more genetic constructs that engender expression of polypeptides as described herein. In certain preferred embodiments of the invention, DNAs that comprise cis-acting transcription controls for expressing a polypeptide operably linked to a region encoding the polypeptide are highly preferred. In other preferred embodiments a multi-gene system directing expression of a polypeptide and containing the DNA sequences coding for such a polypeptide, are highly preferred. Also highly preferred in this regard are single and or multi-gene constructs as described herein, that engender expression of genetically engineered genes for polypeptides. Constructs that comprise operable signal sequences that effectuate transport of the polypeptide product into a targeted compartment of an organism, such as a tissue or fluid, are further preferred in certain embodiments in this regard. Also especially preferred in this regard are constructs that are stably incorporated in the genome of germ line cells of the mature organism and inherited in normal, Mendelian fashion upon reproduction. One or more DNA or RNA:DNA hybrids or the like may be used alone or together to make transgenic organisms useful in the invention as described further below.

Standard, as well as unusual and new techniques for making transgenic organisms generally can be used to make transgenic organisms in accordance with the invention. Useful techniques in this regard include, but are not limited to, those that introduce genetic constructs by injection, infection, transfection—such as calcium phosphate transfection, using cation reagents, using sperm or sperm heads or the like—lipofection, liposome fusion, electroporation, and ballistic bombardment. Useful techniques include both those that involve homologous recombination, which can be employed to achieve targeted integration, and those that do not, such as those disclosed below.

Constructs can be introduced using these and other methods into differentiated cells, such as fibroblast cells, which are capable of being reprogrammed and then cloned, pluripotent cells, totipotent cells, germ line cells, eggs, embryos at the one cell stage, and embryos at several cell stages, among others, to make transgenic organisms of the invention. In these regards, among others, they may be introduced by such methods into pronuclear, nuclear, cytoplasmic or other cell compartments or into extracellular compartments of multicellular systems to make transgenic organisms of the invention.

In a preferred method, developing embryos can be infected with retroviral vectors and transgenic animals can be formed from the infected embryos. In a particularly preferred method DNAs in accordance with the invention are injected into embryos, at the single-cell or several cell stage. In some particularly preferred embodiments in this regard, DNA is injected into the pronucleus of a one-cell embryo. In other preferred embodiments in this regard, DNA is injected into the cytoplasm of a one-cell embryo. In yet another particularly preferred embodiment in this regard, DNA is injected into an early stage embryo containing several cells.

The primary and secondary constructs of the multi-gene system can be introduced into a cell at the same time, however, the optimum ratio of primary construct to secondary construct(s) will need to be determined for optimum expression.

Recombinant variant Factor VIII (rvhFVIII) proteins suitable for expression in milk.

As discussed above, both the secretion of wildtype Factor VIII and its stability in the body fluids of transgenic animals is a problem with respect to suitability for production and levels that are enabling for therapeutic uses of the native Factor VIII protein. In a preferred embodiment, a recombinant variant of human FVIII is designed to give both high secretion efficiency and stability in the milk environment while possessing proper post-translational processing needed for FVIII biological activity for therapeutic purposes. Like human plasma, Factor VIII can be degraded or otherwise inactivated by the proteolytic and metal chelating environment of the milk. Recombinant inactivation resistant Factor VIII molecules have been designed for their resistance to the inactivating environment of human plasma to achieve certain therapeutic advantages (ie., the IR8 molecules of Pipe and Kaufman, PNAS 94, 11851-11856). However, this has not been done for the purposes of expression in the milk environment. Here we refer to recombinant variant human Factor VIII (rvhFVIII) molecules (which offer inactivation resistance to the milk environment (here rvhFVIII molecules that are here also termed “IR8”) and also improvements which also provide an increased secretory efficiency to the trafficking proteins of the Golgi apparatus of the mammary gland of transgenic livestock. In a particular embodiment, we present designs that have these same desirable properties of secretory efficiency and stability in the mammary gland and stability in milk of the pig. This can be achieved by a proper inclusion of certain glycosylation sites which may be in the B-domain, single chain design so as to resist light chain disassociation in the milk environment, and appropriate enzymatic structure for activation and feedback regulation by the human physiology when the rvhFVIII is used as a procoagulant in the context of therapeutic applications. Thus, the rvhFVIII of this invention that have IR8 properties have not only resistance to inactivation by the milk environment but also added molecular features for high secretion efficiency in the mammary gland. Furthermore, while not limited to traditional plasma IR8 designs, the rvhFVIII of this invention that are IR8 in their resistance to inactivation by the milk environment may also have traditional properties of IR8 molecules that have resistance to inactivation by the components of human plasma in the context of Factor VIII therapeutic applications. Although not limiting in scope of this invention, a covalently-linked light chain is one of the engineered design features that yields the stability in chelating environments and subsequent processing of the milk into a milk derivative when using chelating agents.

The following examples are provided merely to illustrate the invention, and are not to be interpreted as limiting the scope of the invention which is described in the specification and appended claims.

### EXAMPLE 1

Construction and preparation of the 4.1 kbp long mouse Whey Acidic Protein (1 mWAP) driven rvhFVIII that is an IR8 construct (WAP6IR8) for microinjection into embryos to make transgenic animals.

The WAP6IR8 construct uses the regulator elements of the mouse WAP gene to express a modified Factor VIII cDNA that has the B domain deleted. Specifically, The 4.1 kbp 1 mWAP promoter described in (Paleyanda et al., Transgene Res., 3 (1994) pp. 335-343) is used to direct expression of an altered cDNA for Factor VIII that has the B domain deleted, referred to as IR8 (Inactivation Resistant FVIII) as referenced in (Pipe and Kaufman, PNAS, 94 (1997). p11852) that is followed by ˜1.6 kbp of mouse Whey Acidic Protein (mWAP) 3′UTR (C. Russell, dissertation “Improvement of Expression of Recombinant Human Protein C in the Milk of Transgenic Mammal Using a Novel Transgenic Construct,” Virginia Polytechnic Institute, Blacksburg, Va. (December 1993)) coding for the polyadenylation signal. Assembly of the WAP6IR8 and its purification for microinjection is by routine recombinant DNA techniques known to the skilled artisan that can be found, for example, in Sambrook et al., MOLECULAR CLONING, A LABORATORY MANUAL, Vol. 1-3 (Cold Spring Harbor Press 1989).

Step 1. Modification of the plasmid pUCWAP6.

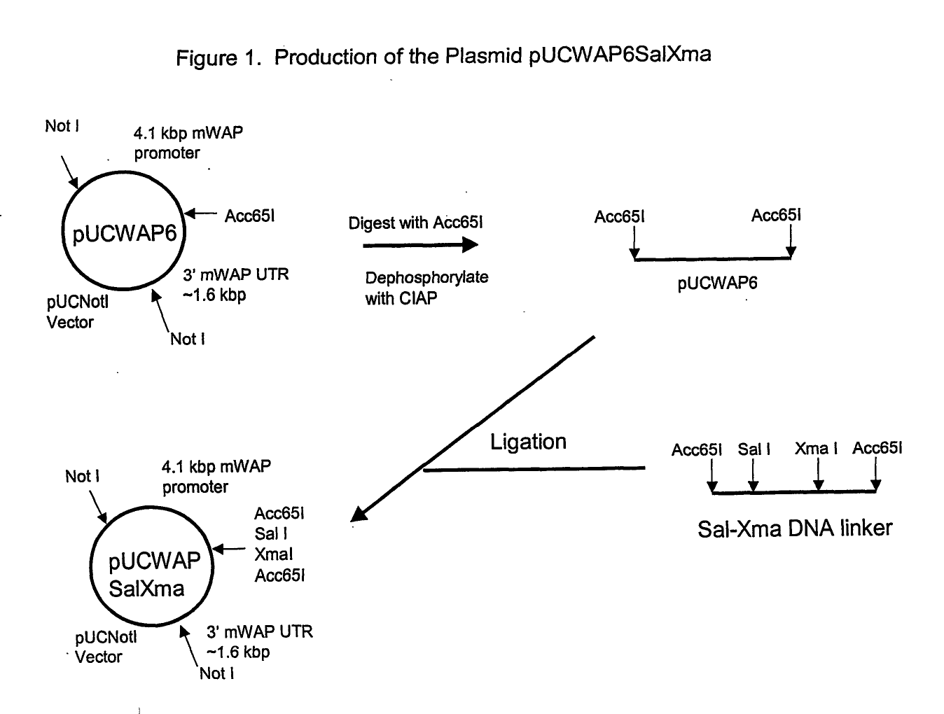

The cassette vector pUCWAP6 described in (S. Butler, thesis “Production and Secretion of Recombinant Human Fibrinogen by the Transgeneic Murine Mammary Gland”, Virginia Polytechnic Institute, Blacksburg, Virginia (May, 1997)) containing the 4.1 kbp 1 mWAP promoter and ˜1.6 kbp mWAP 3′UTR has an unique Kpn I endonuclease site immediately 3′ of the promoter region and 5′ of the 3′UTR which is used for cloning in coding sequences such as cDNAs. Due to an internal Kpn I site in the IR8 cDNA, the pUCWAP6 Kpn I site is changed by adding in a DNA linker containing recognition sequences for Sal I and Xma I endonucleases. Specifically, pUCWAP6 is digested with endonuclease Acc65I (all enzymes are from Invitrogen; Carlsbad, Calif. unless otherwise noted) and dephosphorylated using Calf Intestinal Alkaline Phosphatase (CIAP) per manufacture's instructions (Promega; Madison, Wis.) followed by agarose gel purification using an UltraClean 15 kit (MoBio; Solana Beach, Calif.). Two oligos SalXmaS (5′-pgtaccgtcgacaattcccgggg) and SalXmaA (5′-pgtacccccgggaattgtcgagg) are boiled for 5 min and allowed to cool to room temperature producing the Sal-Xma DNA linker. The dephosphorylated pUCWAP6 is ligated with the Sal-Xma linker and the ligation mixture is then used to transform competent E. coli cells. Transformants containing the modified pUCWAP6 plasmid are screened by digestion of their corresponding plasmids with Xma I endonuclease and observing a band size of ˜8.4 kbp after agarose gel electrophoresis. Plasmids of ˜8.4 kbp are selected and sequenced across the linker junction using primer 1 mWAP for (5′-atgcatcccagacactcaga) to determine the orientation of the linker. Plasmids with the orientation of 4.1 kbp 1 mWAP promoter—Sal I—Xma I—m WAP 3′UTR are deemed correct and are identified as pUCWAP6SalXma (FIG. 1).

Step 2. Production of the 5′ and 3′ ends of IR8.

To facilitate cloning into the pUCWAP6SalXma cassette vector described above, both the 5′ and 3′ ends of the IR8 cDNA are modified. The plasmid p90/b/73 R336I/R562K/R740A containing the IR8 cDNA kind gift from Randal Kaufman, University of Michigan) is used as a template for Polymerase hain Reaction (PCR) to generate the 5′ IR8 fragment. This fragment contains a unique Sal I endonuclease site 5′ of the first prepropeptide codon (ATG) and extending to the naturally occurring Spe I endonuclease site. Primers for PCR are: 5′IR8for (5′-gtcgacatgcaaatagagctctccacctg) and 5′IR8rev (5′-catactagtagggctccaatgagg) and produce an ˜530 bp product. This PCR product is cloned into the pCR4 plasmid (Invitrogen) using the TOPO kit (Invitrogen) per manufacture's instructions and propagated in E. coli. The plasmid containing the insert is called pCR5′IR8. The 3′ IR8 fragment contains 3′IR8 coding sequences though the naturally occurring BspE I site to the natural stop site, TGA. Unique Spe I and XmaI recognition sequences are placed on the 5′ and 3′ ends respectively. The plasmid p90/b/73 R336I/R562K/R740A containing the IR8 cDNA is used as template for PCR to generate the 3′ IR8 fragment. Primers for PCR are: 3′IR8for (5′-agctagtccagacttcattattccgga) and 3′IR8rev (5′-cccgggtcagtagaggtcctgtgggt) and produce an ˜860 bp product. This PCR product is cloned into the pCR4 plasmid (Invitrogen) using the TOPO kit (invitrogen) per manufacture's instructions and propagated in E. coli. The plasmid containing the insert is called pCR3′IR8.

Step 3. Production of pUCWAP6IR8

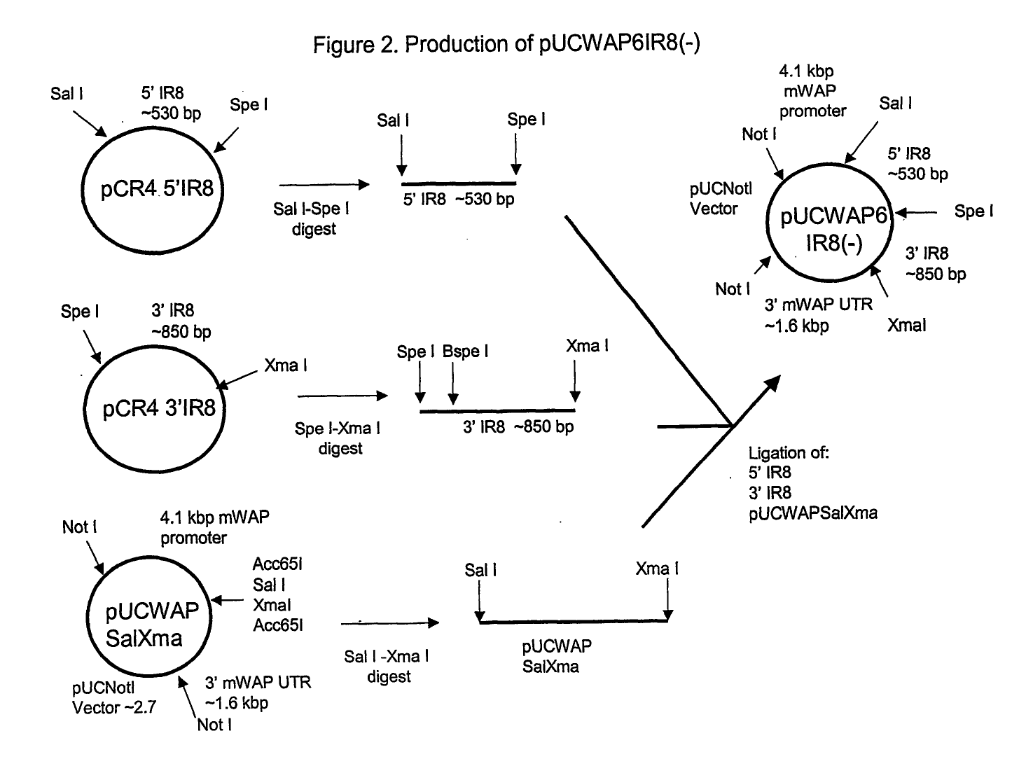

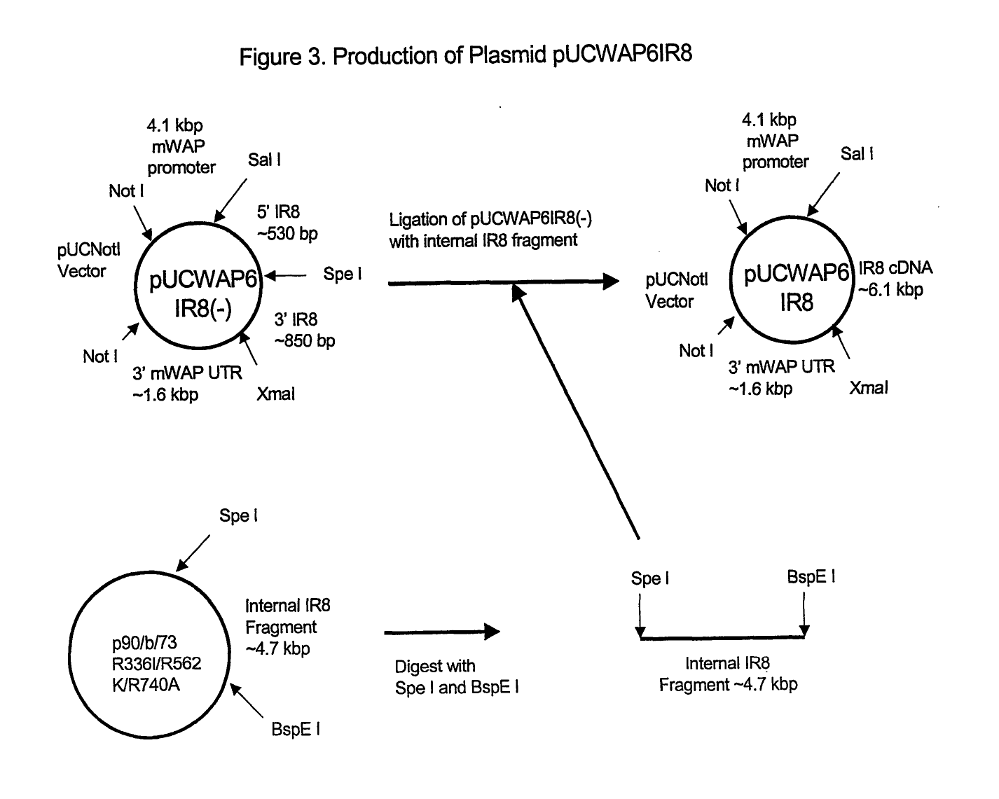

The plasmid pUCWAP6SalXma is cut with the endonucleases Sal I and Xma I and gel purified. This is ligated with the gel purified 5′IR8 fragment removed from pCR5′IR8 by Sal I and Spe I digestion and the gel purified 3′IR8 fragment that is removed from pCR3′IR8 by Spe I and Xma I digestion. The ligation mixture is then used to transform competent E. coli with transformants screened by digestion of their corresponding plasmids with Sal I and Xma I endonucleases and observance of an ˜1.4 and ˜8.4 kbp band after agarose gel electrophoresis. A plasmid producing the ˜1.4 kbp band is deemed correct and designated pUCWAP6IR8(−), (FIG. 2). The plasmid p90/b/73 R336I/R562K/R740A is digested with the endonucleases Spe I and BspEI to release the ˜4.7 kbp internal fragment of IR8. This internal fragment is gel purified and ligated into pUCWAP6IR8(−) that is digested with Spe I and BspEI and gel purified. The ligation mixture is then used to transform competent E. coli with transformants screened by digestion of their corresponding plasmids with Sal I and Xma I endonucleases and observance of an ˜6.1 kbp band after agarose gel electrophoresis. A plasmid producing the ˜6.1 kbp band is deemed correct and designated pUCWAP6IR8 (FIG. 3).

Step 4. Preparation of 4.1 kbp WAP driven IR8 construct (WAP6IR8) for microinjection.

The DNA fragment used for microinjection of early stage embryos was prepared by endonuclease digestion of pUCWAP6IR8 with the enzyme Not I followed by separation from bacterial elements by agarose gel electrophoresis. The ˜11.9 kbp fragment is excised from the gel and purified, followed by ethanol precipitation and suspension in TE (10 mM Tris pH 7.4,1 mM EDTA). The fragment is further purified by subjecting the fragment to ultracentrifugation through a standard NaCl gradient. DNA concentration is determined by agarose gel electrophoresis by staining with ethidium bromide and comparing the fluorescent intensity of an aliquot of the DNA with the intensity of standards. Samples are then adjusted to 5 μg/ml.

### EXAMPLE 2

Production of WAP6IR8 Transgenic Mice.

Step 1. Transgenic mice are produced essentially as described by Hogan et al., Manipulating the Mouse Embryo, Cold Spring Harbor Press, (1986), which is hereby incorporated by reference. That is, glass needles for micro-injection were prepared using a micropipet puller and microforge. Injections are performed using a Nikon microscope having Hoffman Modulation Contrast optics, with Narashigi micromanipulators and a pico-injector driven by N2 (Narashigi). Fertilized mouse embryos are surgically removed from oviducts of superovulated female CD-1 mice and placed into M2 medium. Cumulus cells are removed from the embryos with hyaluronidase at 300 μg/ml. The embryos are then rinsed in new M2 medium, and stored at 37 degrees centigrade prior to injection. Stock solutions containing about 5 μg/ml of the above described DNA are prepared and microinjected into non-pronuclear stage mouse embryos. After injecting the DNA, embryos are implanted into avertin-anesthesized CD-1 recipient females made pseudo-pregnant by mating with vasectomized males. About 25-30 microinjected mouse embryos per recipient are transferred into pseudopregnant females.

Step 2. DNA from mice born after embryo transfer is isolated by digesting tissue in (50 mM Tris-HCl, 0.15 M NaCl, 1 M Na₂ ClO₄, 10 mM EDTA, 1% sodium dodecylsulfate, 1% 2-mercaptoethanol, 100 ug/ml proteinase K, pH 8.0). 7501 of lysate was extracted with 250 1 chloroform/phenol (1:1) followed by precipitation with isopropanol 0.7 volumes, washed in 70% ethanol and dried. DNA is suspended in TE (10 mM Tris-HCl and 1 mM EDTA pH 8.0). Mice produced after embryo transfer of microinjected embryos are screened by Southern analysis. To confirm the presence of the IR8 cDNA, 10 μg of DNA isolated from tail tissue is digested with the endonucleases SaI I and Xma I an subjected to agarose gel electrophoresis and transferred to a nylon membrane. The membrane is probed with a ³²P labeled DNA fragment of the IR8 cDNA consisting of the Sal I to Spe I fragment (˜530 bp). Hybridization was carried out at 68° C. for 4 hours using Quick Hyb (Stratagene; LaJolla, Calif.). Following standard washing methods, the membrane is subjected to autoradiography (−70° C.) for a period of 24 hours. Observance of a ˜6.1 kbp band indicates the presence of the transgene.

### EXAMPLE 3

Production of WAP6IR8 Transgenic Pigs.

Step 1. Pig embryos are recovered from the oviduct, and placed into a 1.5 ml microcentrifuge tube containing approximately 0.5 ml embryo transfer media (Beltsville Embryo Culture Medium). Embryos are centrifuged for 12 minutes at 16,000×g RCF (13,450 RPM) in a microcentrifuge (Hermle, model Z231). The embryos are then removed from the microcentrifuge tube with a drawn and polished Pasteur pipette and placed into a 35 mm petri dish for examination. Embryos are then placed into a microdrop of media (approximately 100 μl) in the center of the lid of a 100 mm petri dish, and silicone oil is used to cover the microdrop and fill the lid to prevent media from evaporating. The petri dish lid containing the embryos is set onto an inverted microscope (Carl Zeiss) equipped with both a heated stage and Hoffman Modulation Contrast optics (200× final magnification). A finely drawn (Kopf Vertical Pipette Puller, model 720) and polished (Narishige microforge, model MF-35) micropipette is used to stabilize the embryos while about 1-2 picoliters of stock solution (5 μg/ml) of the above described DNA is microinjected into the non-pronuclear stage pig embryos using another finely drawn micropipette. Embryos surviving the microinjection process as judged by morphological observation are loaded into a polypropylene tube (2 mm ID) for transfer into the recipient pig. About 40-50 microinjected embryos are transferred into each hormonally synchronized surrogate mother recipient female pig.

Step 2. Pigs produced after embryo transfer of microinjected embryos are screened by Southern analysis. Screening for the WAP6IR8 construct, 10 μg of DNA isolated from tail tissue (as described above for mice) is digested with the endonucleases Sal I and Xma I and subjected to agarose gel electrophoresis and transferred to a nylon membrane. The membrane is probed with a ³²P labeled DNA fragment of the IR8 cDNA consisting of the Sal I to Spe I fragment (˜530 bp). Hybridization is carried out at 68° C. for 4 hours using Quick Hyb (Stratagene; LaJolla, Calif.). Following standard washing methods, the membrane is subjected to autoradiography (˜70° C.) for a period of 24 hours. Observance of a ˜6.1 kbp band indicates the presence of the transgene.

### EXAMPLE 4

Collection and storage of milk from WAP6IR8 Transgenic mice.

Mouse milk is collected and stored as well described in the prior art (Velander et al., Annals of the New York Academy Sciences, 665 (1992) 391-403.)

### EXAMPLE 5

Collection and storage of milk from WAP6IR8 Transgenic Pigs.

Lactating sows are injected intramuscularly with 30-60 IU of oxytocin (Vedco Inc., St. Joseph, Mo.) to stimulate milk let-down. Letdown occurs two to five minutes after injection. Pigs are milked by hand during the course of this study. Immediately after collection the milk is diluted 1:1 with 200 mM EDTA, pH 7.0 to solubilize the caseins and then frozen. Small aliquots (about one milliliter) of the milk/EDTA mixture are taken and centrifuged for approximately 30 minutes at 16000×g at 4° C. The fat layer is separated from the diluted whey fraction, and the diluted whey fraction is used for all further assays.

### EXAMPLE 6

Detection of high levels of recombinant variant human Factor VIII (rvhFVIII) in milk of transgenic mice and transgenic pigs

Step 1. Detection of rvhFVIII by ELISA.

Data from milk samples from the entire lactation of transgenic mice and that of transgenic pigs described in examples 4 and 5) respectively that are processed to diluted whey samples are multiplied by a factor of 1.9 to account for dilution with EDTA and subsequent removal of milk fat. Amounts of Factor VIII antigen in milk are measured by polyclonal ELISA. Briefly, Immulon II microtiter plates (Fisher Scientific, Pittsburgh) are coated overnight with 100 μl/well of 1:1000 rabbit anti-human Factor VIII in 0.1 M NaHCO₃, 0.1 M NaCl, pH 9.6 at 4° C. The wells are washed with TBS-Tween (TBST, 25 mM Tris, 50 mM NaCl, 0.2% Tween 20, pH 7.2), and then blocked for 30 minutes with TBS/0.1% BSA at room temperature. Samples and human Factor VIII standard derived from plasma in the TBS-BSA dilution buffer are added in triplicate to the wells (I100 μl/well) and incubated at 37° C. for 30 minutes. The wells are then washed and blocked for another 10 minutes at room temperature. Sheep anti-human Factor VIII (1:1000 in TBS-BSA), is then incubated in the wells for 30 minutes at 37° C., followed by anti-sheep IgG/HRP (Sigma, St. Louis). Bound chromophore is detected with OPD substrate (Abbott, Chicago) at 490 nm using an EL308 Bio-Tek Microplate reader. Daily expression levels of the recombinant variant human Factor VIII are about greater than 20 μg/ml milk and this is detected throughout about a 21 day lactation in mice and about a 50-60 day lactation in pigs. Variants having all or part of the B-domain are detected by this ELISA method.

Step 2. Detection of high levels of recombinant variant human Factor VIII (rvhFVIII) in milk of transgenic mice and pigs by Western Blot Analysis.

Recombinant variant human Factor VIII (rvhFIX) also is examined using Western Blot Analysis. Daily samples of EDTA-diluted whey as prepared above and taken from transgenic short WAP-FIXcDNA pigs are electrophoresed on 8-16% SDS gels (Novex, San Diego). Approximately 125 ng of recombinant human Factor IX (as determined by polyclonal ELISA) and human Factor VIII standard derived from plasma are loaded in each lane. A total of 25 μg of total protein from a pool of non-transgenic (NTG) whey is loaded on the gels. After electrophoresis, proteins are transferred overnight to PVDF membranes (Bio Rad). The membranes are washed for 30 minutes in TBST, blocked with TBS/0.05% Tween 20/0.5% Casein (TBST-Casein). The membranes are developed with rabbit anti-Factor IX (Dako) (1:1000 in TBST-Casein for 45 minutes at 37° C.), followed by anti-rabbit IgG/HRP (Sigma) (1:1000 in TBST-Casein for 45 minutes at 37° C.), and the DAB metal enhanced staining (Pierce). Molecular weight markers are purchased from Bio-Rad. The presence of about greater than 20 ug/ml of structurally intact rvhFVIII in the milk of transgenic mice and transgenic pigs is detected by the Western Blot Analysis method.

Step 3. Purification and biological activity by APTT of rvFVIII in transgenic mice and pigs.

The immunoaffinity chromatographic process well described by Paleyanda et al., Nature Biotechnology, 15 (1997) 971-975 is applied to the milks of transgenic mice and pigs containing rvhFVIII. The one stage clotting assay (APTT) of the same authors is used to assess the specific procoagulant activity of the immunopurified rvhFVIII.

The specific activity is found to be 50% or greater of that of Factor VIII derived from human plasma.

### EXAMPLE 7

A milk derivative containing high levels of rvhFVIII suitable for therapeutic applications.

A milk derivative concentrate of a recombinant variant human Factor VIII useful for oral delivery of rvhFVIII is made from the milk of a transgenic animal containing a transgene composed of the 4.1 kbp mouse whey acidic protein promoter (WAP), a DNA encoding sequence for rvhFVIII, and a 1.4 kb fragment of the 3′UTR of WAP. The expression level of rvhFVIII is about 20 ug/ml or greater. Greater than about 50% of the rvhFVIII is biologically active as a procoagulant. The skim milk is treated with a chelating agent such as 100 mM EDTA pH 7.5 or 100 mM Sodium Citrate pH 6.5 to clarify the milk of casein micelles. The clarified whey is passed over a DEAE-Sepharose or DEAE-Cellulose chromatographic column and the rvhFVIII is adsorbed. This adsorbed rvhFVIII is selectively desorbed from the anion exchange column using 50-250 mM Ca²⁺Tris-buffered-saline 150 mM NaCl (TBS) linear gradient. This eluted fraction of rvhFVIII containing selected, highly biologically active fractions of rvhFVIII is useful for oral delivery of rvhFVIII for therapeutic treatment of hemophilia A patients is passed through a 0.2 micron filter top remove bacterial contamination and then lyophilized to a powder. The rvhFVIII in the DEAE-column eluate has a composition that is volume reduced and concentrated by 25 to 50-fold over that of starting skim milk.

### EXAMPLE 8

Corrected bleeding times by oral delivery of a milk derivative containing high levels of rvhFVIII made by transgenic animals

The lyophilized powder of example 6 is reconstituted with aqueous containing ordinary bovine milk cream such as to restore the volume to 25 to 100-fold concentrate over that of the original whey. The mixture is fed to hemophilia type A mice shortly after their first meal post sleep. The bleeding time by measured tail incision is measured 12 hours later. The corrected bleeding time is about 5-7 minutes as compared to about 10 to 15 minutes for a control hemophiliac mouse who was not fed the rvhFVIII milk concentrate and about 5 minutes for a normal mouse with normal hemostasis.

### EXAMPLE 9

A milk derivative containing high levels of rvhFVIII that is an IR8 design suitable for therapeutic applications (rvhFVIII-IR8).

A milk derivative concentrate of recombinant variant human Factor VIII useful for oral delivery of rvhFVIII is made from the milk of a transgenic animal containing a transgene composed of the 4.1 kb mouse whey acidic protein promoter (WAP), the cDNA encoding mutant human FVIII-IR8, and a 1.4 kb fragment of the 3′UTR of WAP. The expression level is greater than about 20 ug/ml of rvhFVIII versus only less than 0.1 ug/ml in the milk of transgenic mice expressing the full length cDNA of wildtype rhFVIII. Greater than about 50% of the rvhFVIII is biologically active. The skim milk is treated with a chelating agent such as 100 mM EDTA pH 7.5 or 100 mM Sodium Citrate pH 6.5 to clarify the milk of casein micelles. The clarified whey is passed over a dextran sulfate(DS)-Sepharose 4B chromatographic column and the rhFVIII is adsorbed. This adsorbed rhFVIII-IR8 is selectively desorbed from the anion exchange column using Tris-buffered-saline (120 mM NaCl; pH 7); at 175 mM CaCl2 (TBS). This eluted fraction of rvhFVIII-IR8 containing selected, highly biologically active fractions of rvhFVIII-IR8 is useful for oral delivery of FVIII for therapeutic treatment of hemophilia A patients is pass through a 0.2 micron filter top remove bacterial contamination and then lyophilized to a powder. The rvhFVIII in the DS-Sepharose column eluate has a composition that is volume reduced and concentrated by 25- to 100-fold over that of starting skim milk. The concentrated eluate is then lyophilized.

### EXAMPLE 10

Corrected bleeding times by oral delivery of a milk derivative containing high levels of rvhFVIII-IR8 made by transgenic animals

The lyophilized powder of example 9 is reconstituted with aqueous containing ordinary bovine milk cream such as to restore the volume to 25 to 100-fold concentrate over that of the original whey. Less than 1 ml of the mixture is fed to hemophilia type A mice shortly after their first meal post sleep. The bleeding time by measured tail incision is measured 12 hours later. The corrected bleeding time is about 5-7 minutes as compared to about 10 to 15 minutes for a control hemophiliac mouse who was not fed the rvhFVIII milk concentrate and about 5 minutes for a normal mouse with normal hemostasis.

### EXAMPLE 11

Oral immunotolerization of rvhFVIII by a milk derivative containing rvhFVIII from a transgenic animal.

Mice are fed the reconstituted mixture from example 9, everyday consecutively for one month and after this month, they are sensitized with complete Freund's adjuvant and ReFacto, a commercially available recombinant human variant human Factor VIII that is B-domain deleted in its structure and made by Pharmacia, Stockholm Sweden. After 12 days, blood samples from these mice do not respond with the presence of anti-human FVIII antibodies and also does not respond with T-cells which are activated by the presence of human FVIII. Control mice that have not been fed the mixture from example 11 are sensitized with the same adjuvant and human rvhFVIII mixture. After about 12-14 days the blood of these human FIX sensitized control mice exhibit a strong immunological response consisting of both anti-human FIX antibodies and T-cells that are activated by the presence of rvhFVIII.

What is claimed is:

1. A non-human transgenic mammal containing an exogenous DNA molecule stably integrated in its genome, wherein said exogenous DNA molecule comprises:
(a) 5′ regulatory sequences of a mammary gland-specific gene including a promoter; and
(b) a recombinant variant human Factor VIII-encoding DNA sequence that encodes a signal peptide sequence, followed by a Factor VIII propeptide sequence, a modified Factor VIII sequence in a 5′ to 3′ direction containing a Factor VIII secretion signal motif that is efficient in the mammary epithelia, wherein said trafficking sequences are effective in directing the secretion of said Factor VIII into the milk of said transgenic mammal and wherein said Factor VIII sequence consists of a modified encoding sequence of Factor VIII that is engineered for improved stability in milk; and
(c) 3′ regulatory sequences from a mammary gland-specific gene or 3′ regulatory sequences active in a mammary gland wherein said 5′ and said 3′ regulatory sequences are operatively linked to said Factor VIII-encoding DNA sequence.

2. The non-human transgenic mammal of claim 1, wherein said promoter is selected from the group consisting of rodent whey acidic protein (WAP) promoters, short α-casein promoter, short β-casein promoter, short kappa-casein promoter, long α-casein promoter, long β-casein promoter, long kappa-casein promoter, α-lactalbumin promoter, lactoferrin promoter and β-lactoglobulin promoter.

3. The non-human transgenic mammal of claim 2, wherein said FVIII gene sequence codes for a modified FVIII having a modified B-domain to improve secretion and is secreted as a single polypeptide to improve stability in the milk relative to activation, inactivation and degradation.

4. The non-human transgenic mammal of claim 3, wherein said transgenic mammal is selected from the group consisting of mice, rats, rabbits, pigs, sheep, goats and cows.

5. The non-human transgenic mammal of claim 4, wherein said long WAP promoter is the 4.1 kb NotI-KpnI promoter or the 4.2 kb Sau3A-KpnI promoter of the mouse WAP gene.

6. The non-human transgenic mammal of claim 5 said transgenic mammal is a pig.

7. The non-human transgenic mammal of claim 4, wherein said FVIII-encoding DNA molecule similar to IR8 but has a portion of the B-domain necessary for improved secretion.

8. The non-human transgenic mammal of claim 3, wherein said Factor VIII-encoding DNA molecule further comprises an intron region that is not an intron, region of the Factor VIII gene.

9. The non-human transgenic mammal of claim 8, wherein said Factor VIII-encoding DNA molecule ether comprises the truncated intron I from human Factor IX and the encoding region of a Factor VIII variant.

10. The non-human transgenic mammal of claim 9, wherein said Factor VIII-encoding DNA molecule is a modified FVIII variant having a modified B-domain to improve secretion and is secreted as a single polypeptide to improve stability in the milk relative to activation, inactivation and degradation.

11. The non-human transgenic mammal of claim 1, wherein said transgenic mammal is selected from the group consisting of mice, rats, rabbits, pigs, sheep, goats and cows.

12. The non-human transgenic mammal of claim 5 said transgenic mammal is a pig.

13. The non-human transgenic mammal of claim 4, wherein said Factor VIII is a suitably biologically active human Factor VIII variant for therapeutic applications.

14. The non-human transgenic mammal of claim 13, wherein said transgenic mammal secretes from about 20 to about 1000 μg of biologically active human Factor VIII per milliliter milk.

15. The non-human transgenic mammal of claim 13, wherein said transgenic mammal secretes from about 20 to about 500 μg of suitably biologically active human Factor VIII per milliliter milk.

16. The non-human transgenic mammal of claim 13, wherein said transgenic mammal secretes from about 20 to about 300 μg of suitably biologically active human Factor VIII per milliliter milk.

17. The non-human transgenic mammal of claim 13, wherein said transgenic mammal secretes from about 20 to about 100 μg of suitably biologically active human Factor VIII per milliliter milk.

17a. The non-human transgenic mammal of claim 13, wherein said transgenic mammal secretes from about 20 to about 50 μg of suitably biologically active human Factor VIII per milliliter milk.

17b. The non-human transgenic mammal of claim 14, wherein said transgenic mammal is a pig.

18. The non-human transgenic mammal of claim 14, wherein said transgenic mammal is a goat.

19. The non-human transgenic mammal of claim 14, wherein said transgenic mammal is a cow.

20. The non-human transgenic mammal of claim 14, wherein active fractions of human Factor VIII, when purified from the milk of said transgenic mammal, has a specific activity that is at least about 50-500% of that defined for the specific activity of human Factor VIII isolated from human plasma, as determined by an activated partial thromboplastin clotting time assay coagulation assay.

21. The non-human transgenic mammal of claim 20, wherein said transgenic mammal is a pig.

22. The non-human transgenic mammal of claim 20, wherein said transgenic mammal is a goat.

23. The non-human transgenic mammal of claim 20, wherein said transgenic mammal is a cow.

24. A process for producing a Factor VIII variant comprising:
(a) providing a non-human transgenic mammal having integrated into its genome an exogenous DNA molecule, wherein said exogenous DNA molecule comprises:
(1) 5′ regulatory sequences of a mammary gland-specific gene including a promoter;
(2) a recombinant variant human Factor VIII-encoding DNA sequence that encodes a signal peptide sequence, followed by a Factor VIII propeptide sequence, a modified Factor VIII sequence in a 5′ to 3′ direction containing a Factor VIII secretion signal motif that is efficient in the mammary epithelia, wherein said trafficking sequences are effective in directing the secretion of said Factor VIII into the milk of said transgenic mammal and wherein said Factor VIII sequence consists of a modified encoding sequence of Factor VIII that is engineered for improved stability in milk; and
(b) 3′ regulatory sequences from a mammary gland-specific gene or 3′ regulatory sequences active in a mammary gland wherein said 5′ and said 3′ regulatory sequences are operatively linked to said Factor VIII-encoding DNA sequence; and
(c) allowing said DNA sequences encoding said Factor VIII to be expressed and said Factor VIII to be secreted into the milk of said transgenic mammal; and
(d) collecting said milk from said mammal; and
(e) processing said Factor VIII from said milk to produce a milk derivative that is appropriate for therapeutic purposes.

25. The process of claim 24, wherein said promoter is selected from the group consisting of rodent whey acidic protein (WAP) promoters, short α-casein promoter, short β-casein promoter, short kappa-casein promoter, long α-casein promoter, long β-casein promoter, long kappa-casein promoter, α-lactalbumin promoter, lactoferrin promoter and β-lactoglobulin promoter.

26. The process of claim 25 wherein wherein said FVIII gene sequence codes for a modified FVIII having a modified B-domain to improve secretion and is secreted as a single polypeptide to improve stability in the milk relative to activation, inactivation and degradation.

27. The process of claim 26, wherein said transgenic mammal is selected from the group consisting of mice, rats, rabbits, pigs, sheep, goats and cows.

28. The process of claim 27, wherein said long WAP promoter is the 4.1 kb NotI-KpnI promoter or the 4.2 kb Sau3A-KpnI promoter of the mouse WAP gene.

29. The process of claim 27, the non-human transgenic mammal of claim 5 said transgenic mammal is a pig.

30. The process of claim 27, wherein said Factor VIII-encoding DNA molecule is a modified FVIII having a modified B-domain to improve secretion and is secreted as a single polypeptide.

31. The process of claim 27, wherein said FVIII-encoding DNA molecule similar to IR8 but has a portion of the B-domain necessary for improved secretion.

32. The process of claim 27, wherein said Factor VIII is a modified human Factor VIII useful for treating hemophilia A.

33. The process of claim 27, wherein said transgenic mammal secretes from about 20 to about 1000 μg of human Factor VIII per milliliter milk.

34. The process of claim 27, wherein said transgenic mammal secretes from about 20 to about 500 μg of human Factor VIII per milliliter milk

35. The process of claim 27, wherein said transgenic mammal secretes from about 20 to about 300 μg of human Factor VIII per milliliter milk

36. The process of claim 27, wherein said transgenic mammal secretes from about 20 to about 100 μg of human Factor VIII per milliliter milk.

37. The process of claim 27, wherein said transgenic mammal secretes from about 20 to about 50 μg of human Factor VIII per milliliter milk.

38. The process of claim 33, wherein said transgenic mammal is a pig.

39. The process of claim 33, wherein active fractions of the modified Factor VIII when purified from the milk of said transgenic mammal, have a specific activity that is at least about 50-500% of that defined for the specific activity of human Factor VIII isolated from human plasma, as determined by an activated partial thromboplastin clotting time assay coagulation assay.

40. The process of claim 34, wherein active fractions of modified Factor VIII purified from the milk of said transgenic mammal have a specific activity that is at least about 50-500% of that defined for the specific activity of human Factor VIII isolated from human plasma.

41. The process of claim 35, wherein active fractions of human Factor VIII purified from the milk of said transgenic mammal have a specific activity that is at least about 50-500% of that defined for the specific activity of human Factor VIII isolated from human plasma.

42. The process of claim 36, wherein active fractions of human Factor VIII purified from the milk of said transgenic pig have a specific activity that is at least about 50-500% of that defined for the specific activity of human Factor VIII isolated from human plasma.

43. The process of claim 39, wherein said transgenic mammal is a pig.

44. A method of treating a patient having hemophilia A comprising administering to said patient a hemophilia A symptom preventing or ameliorating amount of Factor VIII produced by the transgenic non-human mammal of claim 1 and a pharmaceutically acceptable carrier.

45. The method of treating a patient having hemophilia A according to claim 44 where method is oral delivery of Factor VIII.

46. The method of treating a patient having hemophilia A according to claim 44 where method is oral tolerization of Factor VIII.

47. The method of treating a patient having hemophilia A according to claim 44 where method is oral delivery of Factor VIII and milk proteins.

48. The method of treating a patient having hemophilia A according to claim 44 where method is oral tolerization of Factor VIII and milk proteins.

49. The method of treating a patient having hemophilia A according to claim 44 where the pharmaceutically acceptable carrier is a processed derivative of milk.

50. The method of treating a patient having hemophilia A according to claim 49 where method is oral delivery of Factor VIII.

51. The method of treating a patient having hemophilia A according to claim 49 where method is oral tolerization of Factor VIII.

52. The method of treating a patient having hemophilia A according to claim 49 where the method is both oral delivery and oral tolerization of Factor VIII.

53. The method of treating a patient having hemophilia A according to claim 49 where method is both oral delivery and oral tolerization of Factor VIII and milk proteins.

54. The method of treating a patient having hemophilia A according to claim 49 where method is oral tolerization of Factor VIII derived from recombinant cell culture.

55. The method of treating a patient having hemophilia A according to claim 49 where method is oral tolerization of Factor VIII derived from human plasma.

56. A non-human transgenic mammal containing an exogenous DNA molecule stably integrated in its genome, wherein said exogenous DNA molecule comprises:
a mammary gland-specific gene including a promoter; and
a recombinant variant Factor VIII-encoding DNA sequence that encodes an endogenous signal sequence, a Factor VIII pro-sequence and a modified Factor VIII sequence encoding a secretion trafficking motif that is efficient in mammary epithelial cells; and
a 3′ regulatory sequences from a mammary gland-specific gene, which sequences are operatively linked to said Factor VIII-encoding DNA sequence; and
said Factor VIII is stably secreted into the milk at least 20 micrograms Factor VIII per milliliter of milk and is not inactivated or degraded by the milk-environment and therefore useable for Factor VIII therapeutic applications.
